SEQUENCE LISTING (16 sequences)

SEQ ID NO: 1 - DNA, 20 bp, Artificial sequence
  Artificially synthesized PCR primer based on multiple human genes
cacattgggggtaggaacac

SEQ ID NO: 2 - DNA, 20 bp, Artificial sequence
  Artificially synthesized PCR primer based on multiple human genes
accactcgtacttgggatgc

SEQ ID NO: 3 - DNA, 20 bp, Artificial sequence
  Artificially synthesized PCR primer based on multiple human genes
ctaccaaaatgaccgcacct

SEQ ID NO: 4 - DNA, 20 bp, Artificial sequence
  Artificially synthesized PCR primer based on multiple human genes
cacgaactgaagagcatcca

SEQ ID NO: 5 - DNA, 20 bp, Artificial sequence
  Artificially synthesized PCR primer based on multiple human genes
caaggaaggttggcatttgt

SEQ ID NO: 6 - DNA, 20 bp, Artificial sequence
  Artificially synthesized PCR primer based on multiple human genes
aggtaacgccaggaattgca

SEQ ID NO: 7 - DNA, 20 bp, Artificial sequence
  Artificially synthesized PCR primer based on multiple human genes
gctgagctcaaaccctggta

SEQ ID NO: 8 - DNA, 20 bp, Artificial sequence
  Artificially synthesized PCR primer based on multiple human genes
ccggactccgcaaagtctaa

SEQ ID NO: 9 - DNA, 20 bp, Artificial sequence
  Artificially synthesized PCR primer based on multiple human genes
caaggaaggttggcatttgt

SEQ ID NO: 10 - DNA, 20 bp, Artificial sequence
  Artificially synthesized PCR primer based on multiple human genes
cctttcagtcctttgcaagc

SEQ ID NO: 11 - DNA, 20 bp, Artificial sequence
  Artificially synthesized PCR primer based on multiple human genes
ggctggtctgcttgagaaac

SEQ ID NO: 12 - DNA, 20 bp, Artificial sequence
  Artificially synthesized PCR primer based on multiple human genes
cttttcccacagaccttgga

SEQ ID NO: 13 - DNA, 20 bp, Artificial sequence
  Artificially synthesized PCR primer based on multiple human genes
tggtgtgcagggaattacaa

SEQ ID NO: 14 - DNA, 20 bp, Artificial sequence
  Artificially synthesized PCR primer based on multiple human genes
atccctgctgtgggtacaag

SEQ ID NO: 15 - DNA, 20 bp, Artificial sequence
  Artificially synthesized PCR primer based on multiple human genes
acccagaagactgtggatgg

SEQ ID NO: 16 - DNA, 20 bp, Artificial sequence
  Artificially synthesized PCR primer based on multiple human genes
cacattgggggtaggaacac

(19) United States
(12) Patent Application Publication
(10) Pub. No.: US 2025/0302903 A1
(43) Pub. Date: Oct. 2, 2025

(54) COMPOSITION FOR PREVENTION AND TREATMENT OF MUSCLE DISEASE, CONTAINING MAGNOLIA OFFICINALIS EXTRACT AS ACTIVE INGREDIENT

(71) Applicant: TWINPIGBIOLAB INC., Seoul (KR)

(72) Inventors: Min Woo HONG, Gwangju (KR); Ik Hwan HAN, Namyangju-si (KR); Il Seob CHOI, Seoul (KR); Na Ri CHA, Mokpo-si (KR); Woo Jin KIM, Seoul (KR); Sun Kwang KIM, Seoul (KR); Hyun Su BAE, Seoul (KR)

(73) Assignee: TWINPIGBIOLAB INC., Seoul (KR)

(21) Appl. No.: 18/837,001

(22) PCT Filed: Feb. 9, 2022
(86) PCT No.: PCT/KR2022/001982

§ 371 Date: Aug. 8, 2024

(51) Int. Cl. A61K 36/575; A61K 31/05; A61K 33/243; A61P 21/00; A61P 35/00

(52) U.S. Cl. CPC A61K 36/575; A61K 31/05; A61K 33/243; A61P 21/00; A61P 35/00; A61K 2236/333

(57) ABSTRACT

The present disclosure relates to a pharmaceutical composition for treatment of muscle disease, comprising a Magnolia officinalis extract. The Magnolia officinalis extract contains various ingredients other than magnolol and honokiol, and inhibits cisplatin-induced weight loss, muscle mass reduction, grip strength reduction, and muscle fiber damage, and promotes the repair of damaged muscle fibers through macrophage polarization from M1 to M2 without interfering with an antitumor function of cisplatin, and thus can be effectively used for prevention and treatment of muscle disease, especially, anticancer drug-induced muscle disease.

### CROSS REFERENCE TO RELATED APPLICATIONS

This application is a National Stage of International Application No. PCT/KR2022/001982 filed Feb. 9, 2022.

### INCORPORATION BY REFERENCE OF SEQUENCE LISTING

The content of the electronically submitted sequence listing, file name: Q301024_Substitute Sequence Listing as filed.txt; size: 4,158 bytes; and date of creation: Jun. 17, 2025, filed herewith, is incorporated herein by reference in its entirety.

### TECHNICAL FIELD

The present disclosure relates to a pharmaceutical composition for treatment of muscle disease, containing a Magnolia officinalis extract.

### BACKGROUND ART

Muscle accounts for approximately 40% of the human body, and is essentially required to secure an appropriate amount of muscle to maintain the functional ability of the human body and prevent metabolic diseases. The muscle is broadly divided into smooth muscle, cardiac muscle, and skeletal muscle, and the skeletal muscle accounts for a significant portion of the entire body and facilitates the movement of the skeleton. The skeletal muscle is an organ that occupies the largest part of the human body and accounts for 40% to 50% of the total body weight, and plays an important role in various metabolic functions in the body, including energy homeostasis and heat generation. Human muscles decrease by 1% or more per year after the age of 40, and by the age of 80, approximately 50% of maximum muscle mass is lost, and muscle loss in old age is recognized as the most important factor in reducing an overall physical function.

In muscle disease, impairs walking and movement functions progress gradually due to skeletal muscle weakness to make activities of daily living (ADL) difficult and independent living impossible. In addition, the muscle disease causes cardiopulmonary dysfunction and other complications. Among these, cachexia is a syndrome that is commonly accompanied in chronic diseases such as cancer, tuberculosis, AIDS, and chronic obstructive pulmonary disease, and refers to a catabolic state of the internal metabolism, which shows persistent loss of appetite and weight loss, and is accompanied by malnutrition, metabolic imbalance, and loss of muscle or fat. However, unlike other chronic disease patients, cancer patients have the characteristic of not only suffering from the cachexia but also side effects of various anticancer treatments used to treat cancer. (Fearon K. et al., Lancet Oncol 2011;12(5):489-495).

The cachexia occurs in 50 to 80% of patients with gastrointestinal cancer and lung cancer, and the mortality rate due to cachexia is 20 to 30%. Carcinemia is characterized by weight loss due to muscle loss caused by increased catabolic responses due to inflammatory responses and metabolic changes induced by various cytokines, and occurs when muscle loss causes weight loss of more than 5% within 12 months. These changes lower the response rate to chemotherapy or radiotherapy, make the progression of effective anticancer treatment difficult, reduce the quality of life of patients, and shorten survival. Muscle loss is one of the biggest characteristics of cachexia, and is known to be caused by increased protein catabolismand decreased protein production due to overactivation of various cytokines. The cachexia is a symptom including muscle loss (sarcopenia) and has many overlapping areas. Most patients with cachexia have muscle loss (sarcopenia), but all patients with muscle loss do not show symptoms of cachexia. Clinically, the muscle loss (sarcopenia) may be referred to as a prodromal symptom of cachexia. Inflammatory cytokines that act on cachexia affect insulin and testosterone, which regulate muscle metabolism, to cause abnormalities in muscle protein synthesis (Ryu Seung-wan, J. Clin. Nutr. 2017;9:2-6).

Sarcopenia, one of the characteristics of cachexia, is a disease in which the motor nerves that induce skeletal muscle contraction degenerate to prevent skeletal muscle contraction from progressing, or the expression of proteins involved in muscle contraction within the skeletal muscle is reduced or altered to prevent skeletal muscle contraction from progressing, and in the long term, the motor nerves or skeletal muscles are transformed into fibrous tissue. The sarcopenia is caused by various factors such as aging, hormonal imbalance, malnutrition, lack of physical activity, inflammation, and degenerative diseases, but among them, cancer and anticancer chemotherapy are known to be the main causes. Currently, it is known that exercise, protein, and calorie supplementation are helpful for sarcopenia, but do not help much in patients and the elderly, who account for the majority of sarcopenia patients, and thus sarcopenia therapeutic agents are desperately needed. However, as therapeutic agents currently used for sarcopenia, drugs having a direct effect on improving muscle loss and increasing muscle mass are still in the clinical trial stage, and there are currently no drugs that have been finally approved by the FDA. Therefore, there are some efforts to develop sarcopenia therapeutic agents such as selective androgen receptor modulators, activin receptor antagonists, fast skeletal muscle troponin inhibitors, etc. for treatment of sarcopenia, but the efforts have been currently attempted in early clinical trials. Currently, a method of treating the sarcopenia has been mainly used to suppress muscular atrophy caused by degenerative or progressive mutation of muscle cells, which is a type of sarcopenia. For example, in WO 2007/088123, there is disclosed a therapeutic agent for muscular dystrophy containing a nitroxy derivative as an active ingredient, and in WO 2006/081997, there is disclosed a therapeutic agent for muscular dystrophy containing atraric acid or a derivative thereof as an active ingredient. However, these therapeutic agents containing compounds as active ingredients act not only on skeletal muscles with muscular atrophy, but also on visceral muscles or cardiac muscles unrelated to muscular atrophy, and thus cause various large and small side effects so as not to be used for practical treatment. Meanwhile, since hormonal drugs have significantly reduced side effects compared to compound drugs and are biocompatible due to the nature of hormonal drugs, the development of drugs for treating muscular atrophy or sarcopenia using hormonal drugs is accelerating.

In addition, the muscle atrophy is caused by factors such as damage to muscle tissue due to the absence of mechanical stimulation such as decreased use of muscles, destruction of muscle due to direct injury or physical factors, impaired recovery of muscle cells due to aging, and impairment of muscle use due to damage to nerves that control muscle action (Booth F W., J Appl Physiol Respir Environ Exerc Physiol., 1982). In general, disuse atrophy, which gradually progresses to muscle atrophy, occurs due to loss of muscle strength without using the muscles in the relevant area and surrounding area for a long period of time due to disorders or accidents. The muscle atrophy also occurs in the form of myasthenia gravis caused by disease of the muscle itself, muscular dystrophy: progressive muscular dystrophy, myotonic muscular dystrophy, Duchenne type, Becker type, girdle type, facioscapulohumeral type, inflammation occurring in the muscle itself, spinal muscular atrophy caused by damage to the nerves that control the muscle: Weradnig-Hoffmann type, Kugelberg Welander disease, amyotrophic lateral sclerosis (ALS): Lou Gehrig's disease, sphinobulbar muscular atrophy: Kennedy disease, etc.

Such a muscle loss is the most common symptom in cancer patients and occurs when muscles are exposed to inflammation due to the cancer itself or the toxicity of anticancer drugs. The muscle loss limits the patient's activities and anticancer treatment, greatly reduces quality of life, or even leads to death, and thus studies for controlling the muscle loss are increasing.

Meanwhile, macrophages are polarized into anti-inflammatory M2 macrophages and pro-inflammatory M1 macrophages. Among these, the M1 macrophages produce pro-inflammatory cytokines such as IL-1, TNF-α, and IL-6, cause muscle wasting, and increase protein degradation through muscle fiber lysis, while the M2 macrophages secrete various anti-inflammatory cytokines such as TGF-β and IL-10 to induce muscle recovery and regeneration and promote muscle fiber synthesis. Macrophage-derived IGF-1 expresses high levels of matrix metalloproteinase-8 (MMP-8), CD163, and CD206 to polarize macrophage groups into M2a and M2c phenotypes involved in extracellular matrix remodeling and muscle healing and promote muscle recovery and protect against muscular dystrophy, and thus the balance between M1 and M2 macrophage groups is important for muscle recovery.

### DISCLOSURE

### Technical Problem

An object of the present disclosure is to provide a pharmaceutical composition for prevention or treatment of muscle disease.

Another object of the present disclosure is to provide a pharmaceutical composition for prevention or treatment of anticancer drug side effect-induced disease.

Yet another object of the present disclosure is to provide a food composition for prevention or alleviation of muscle disease.

Yet another object of the present disclosure is to provide a composition for promoting differentiation of anti-inflammatory macrophages.

Yet another object of the present disclosure is to provide a method for treating muscle disease.

Yet another object of the present disclosure is to provide a use for using a pharmaceutical composition for prevention or treatment of muscle disease.

Yet another object of the present disclosure is to provide a method for treating anticancer drug side effect-induced disease.

Yet another object of the present disclosure is to provide a use for use in a preparation of a pharmaceutical composition for prevention or treatment of anticancer drug side effect-induced disease.

### Technical Solution

In order to solve the problem, an aspect of the present disclosure provides a pharmaceutical composition for prevention or treatment of muscle disease comprising a Magnolia officinalis extract as an active ingredient.

Another aspect of the present disclosure provides a pharmaceutical composition for prevention or treatment of anticancer drug side effect-induced disease comprising a Magnolia officinalis extract as an active ingredient.

Yet another aspect of the present disclosure provides a food composition for prevention or alleviation of muscle disease comprising a Magnolia officinalis extract as an active ingredient.

Yet another aspect of the present disclosure provides a composition for promoting differentiation of anti-inflammatory macrophages comprising a Magnolia officinalis extract as an active ingredient.

Yet another aspect of the present disclosure provides a method for treating muscle disease comprising administering a Magnolia officinalis extract in a pharmaceutically effective amount to a subject suffering from the muscle disease.

Yet another aspect of the present disclosure provides a use of a Magnolia officinalis extract for use in the preparation of a pharmaceutical composition for prevention and treatment of muscle disease.

Yet another aspect of the present disclosure provides a method for treating anticancer drug side effect-induced disease comprising administering a Magnolia officinalis extract in a pharmaceutically effective amount to a subject suffering from the anticancer drug side effect-induced disease.

Yet another aspect of the present disclosure provides a use of a Magnolia officinalis extract for use in the preparation of a pharmaceutical composition for prevention and treatment of anticancer drug side effect-induced disease.

### Advantageous Effects

According to the present disclosure, the Magnolia officinalis extract contains various ingredients other than magnolol and honokiol, and inhibits cisplatin-induced weight loss, muscle mass reduction, grip strength reduction, and muscle fiber damage, and promotes the repair of damaged muscle fibers through macrophage polarization from M1 to M2 without interfering with an antitumor function, and thus can be effectively used for prevention and treatment of muscle disease, especially, anticancer drug-induced muscle disease.

### DESCRIPTION OF DRAWINGS

FIG. 1 is a diagram showing HPLC analysis of ingredients of a Magnolia officinalis extract.

FIG. 2 is a diagram confirming weight loss and changes in food uptake by administration of a Magnolia officinalis extract in a cisplatin-induced sarcopenia mouse model:
A: Changes in body weight;
B: Changes in body weight relative to control;
C: Colon length;
D: Average daily food uptake;
Control (PBS): PBS administered control group;
CIS: Cisplatin administered group;
CIS+MAG: Cisplatin and magnolol administered group; and
CIS+M.C: Cisplatin and Magnolia officinalis extract administered group (50, 100, and 200 mg/kg).

FIG. 3 is a diagram confirming an effect of inhibiting muscle wasting by administration of a Magnolia officinalis extract in a cisplatin-induced sarcopenia mouse model:
A: Leg muscle weight;
B: Representative leg images (scale bar 0.5 cm);
C: TA muscle weight;
D: Grip strength measured by behavioral function test in forelimb and all limbs;
E: H&E staining of TA muscle (scale bar 200 μm);
F: Cross-sectional area (CSA) analysis;
Control (PBS): PBS administered control group;
CIS+PBS: Cisplatin and PBS administered group;
CIS+MAG: Cisplatin and magnolol administered group; and
CIS+M.C: Cisplatin and Magnolia officinalis extract administered group (50, 100, and 200 mg/kg).

FIG. 4 is a diagram confirming the regulation of M1 and M2 macrophage polarization in skeletal muscle by a Magnolia officinalis extract in a cisplatin-induced sarcopenia mouse model through mRNA expression of M1-specific gene and M2-specific gene:
A and B: M1 macrophage specific markers;
C to F: M2 macrophage specific markers;
Control (PBS): PBS administered control group;
CIS+PBS: Cisplatin and PBS administered group;
CIS+MAG: Cisplatin and magnolol administered group; and
CIS+M.C: Cisplatin and Magnolia officinalis extract administered group (50, 100, and 200 mg/kg).

FIG. 5 is a diagram of confirming an effect of increasing the number of macrophages and IGF-1 expression by a Magnolia officinalis extract in the TA muscle of a cisplatin-induced sarcopenia mouse model:
A: IHC images showing CD68 (macrophage, green), IGF-1 (red), DAPI (nuclei, blue), and cells (white arrows) expressing both CD68 and IGF-1 identified by merging them in TA muscle (Magnification: 40×, scale bar 50 μm);
B: Numbers of IGF-I, CD68 and DAPI positive cells;
C: IGF-1 mRNA expression level;
D: IGF-1 protein expression level;
Control (PBS): PBS administered control group;
CIS+PBS: Cisplatin and PBS administered group;
CIS+MAG: Cisplatin and magnolol administered group; and
CIS+M.C: Cisplatin and Magnolia officinalis extract administered group (50, 100, and 200 mg/kg).

FIG. 6 is a diagram confirming an effect of increasing a ratio of M2a and M2c macrophages by a Magnolia officinalis extract in a cisplatin-induced sarcopenia mouse model:
A and B: FACS dot plots of CD206 and CD163 in CD11b⁺F4/80⁺ and CD45⁺ cells (CD206⁻CD163⁻: M1 macrophage; CD206⁺CD163⁻: M2a macrophage; and CD206⁺CD163⁺: M2c macrophage);
C to E: Graphs of M1 macrophage, M2a macrophage, and M2c macrophage (expressed as % of CD11b⁺F4/80⁺ cells);
F: CD4⁺/CD8 ratio of CD45⁺ cells in splenocytes;
Control (PBS): PBS administered control group;
CIS+PBS: Cisplatin and PBS administered group;
CIS+MAG: Cisplatin and magnolol administered group; and
CIS+M.C: Cisplatin and Magnolia officinalis extract administered group (50, 100, and 200 mg/kg).

FIG. 7 is a diagram confirming an effect of a Magnolia officinalis extract on the anti-tumor activity of cisplatin:
A: Tumor size;
B: Tumor image;
C: Tumor weight;
Control: Control group;
CIS: Cisplatin administered group; and
CIS+M.C: Cisplatin and Magnolia officinalis extract administered group.

### BEST MODE OF THE INVENTION

Hereinafter, exemplary embodiments of the present disclosure will be described in detail with reference to the accompanying drawings. However, the following exemplary embodiments are presented as examples for the present disclosure, and when it is determined that a detailed description of well-known technologies or configurations known to those skilled in the art may unnecessarily obscure the gist of the present disclosure, the detailed description thereof may be omitted, and the present disclosure is not limited thereto. Various modifications and applications of the present disclosure are possible within the description of claims to be described below and the equivalent scope interpreted therefrom.

Terminologies used herein are terminologies used to properly express preferred exemplary embodiments of the present disclosure, which may vary according to a user, an operator's intention, or customs in the art to which the present disclosure pertains. Therefore, these terminologies used herein will be defined based on the contents throughout the specification. Throughout the specification, unless explicitly described to the contrary, when a certain part “comprises” a certain component, it will be understood to imply the inclusion of stated elements but not the exclusion of any other elements.

Throughout this specification, ‘%’ used to indicate the concentration of a specific material is solid/solid (w/w) %, solid/liquid (w/v) %, and liquid/liquid (v/v) %, unless otherwise stated.

In an aspect, the present disclosure provides a pharmaceutical composition for prevention or treatment of muscle disease containing a Magnolia officinalis extract as an active ingredient.

In an exemplary embodiment, the muscle disease may be muscle disease caused by decreased muscle function, muscle tissue damage, muscle wasting, or muscle degeneration, or may be muscle disease caused by cancer.

In an exemplary embodiment, the muscle disease may be at least one selected from the group consisting of muscular atrophy, myopathy, muscular degeneration, myasthenia, muscular injury, dystrophinopathy, myopathy, muscular dystrophy, cachexia, and sarcopenia.

In an exemplary embodiment, the extract may be extracted with at least one solvent selected from the group consisting of water, organic solvents, subcritical fluids, and supercritical fluids, and the organic solvent may be any one selected from the group consisting of lower alcohols having 1 to 4 carbon atoms, hexane (n-hexane), ether, glycerol, propylene glycol, butylene glycol, ethyl acetate, methyl acetate, dichloromethane, chloroform, ethyl acetate, acetone, methylene chloride, cyclohexane, petroleum ether, benzene and mixed solvents thereof.

In an exemplary embodiment, the Magnolia officinalis extract may be an ethanol extract, and more preferably a 70% ethanol extract.

In an exemplary embodiment, magnolol and honokiol may be contained in a ratio of 2:1 to 4:1 (w/w).

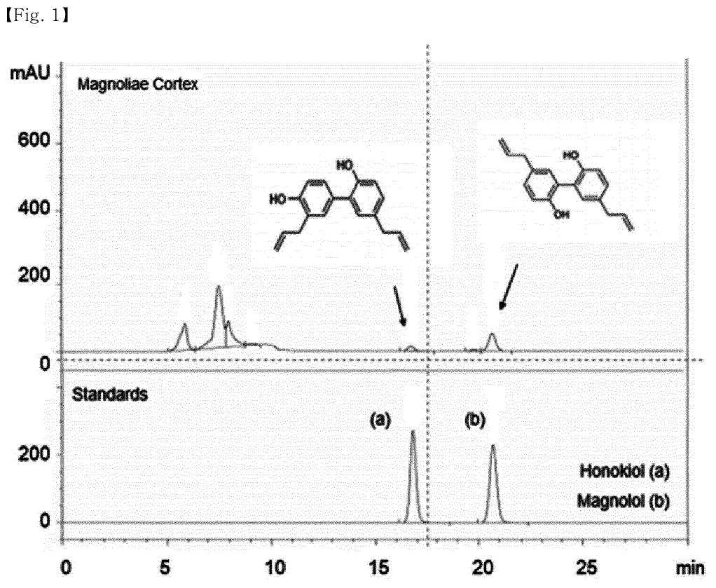

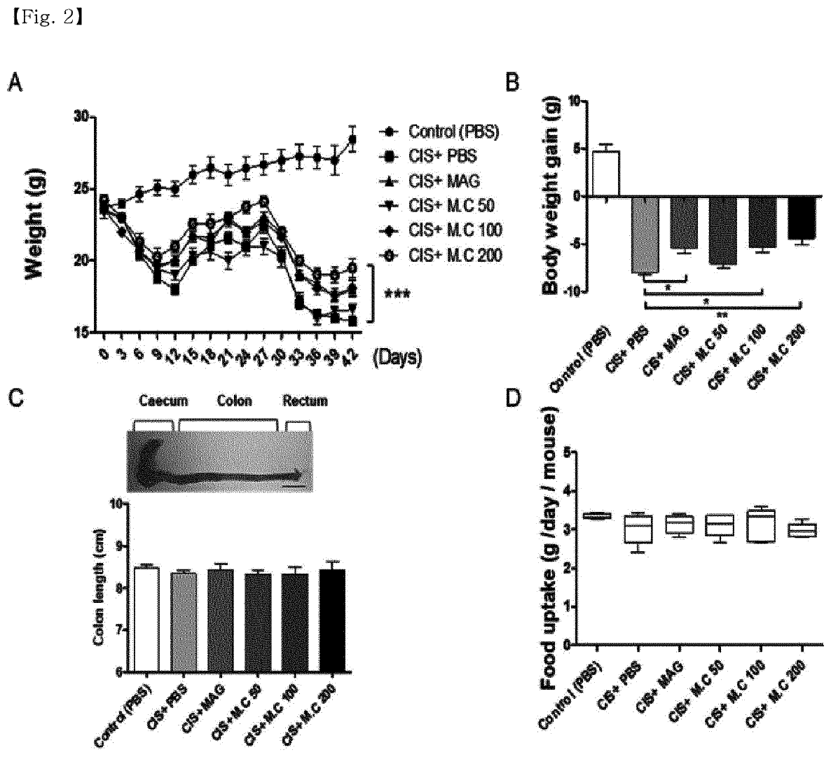

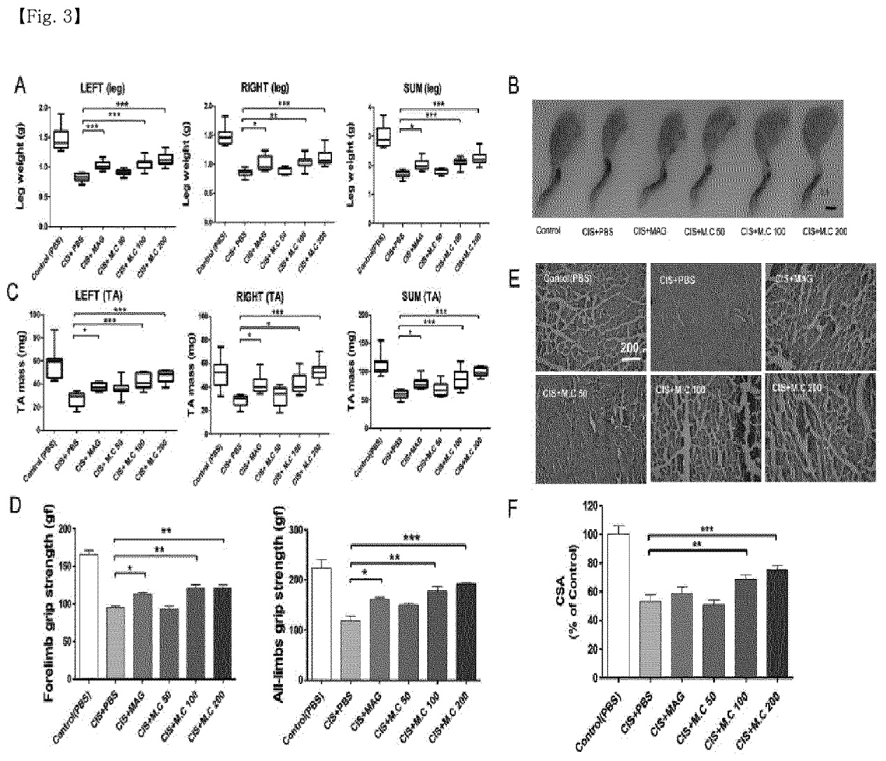

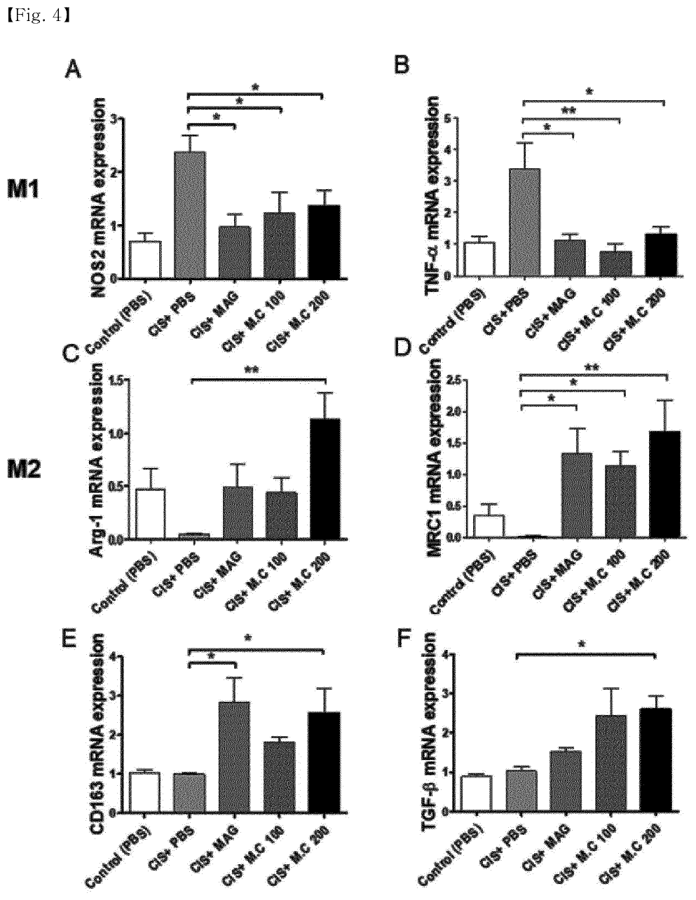

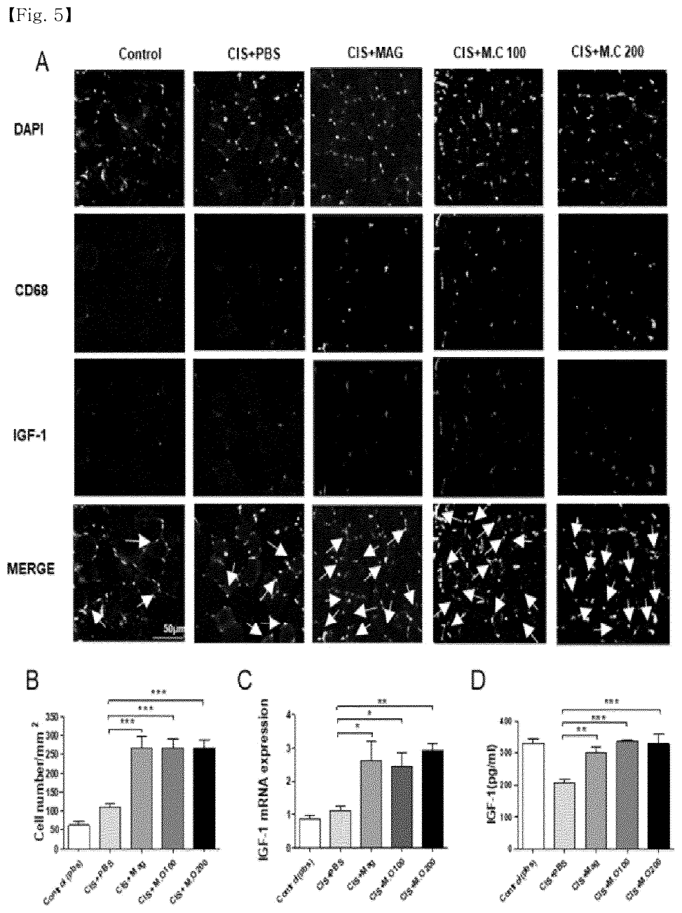

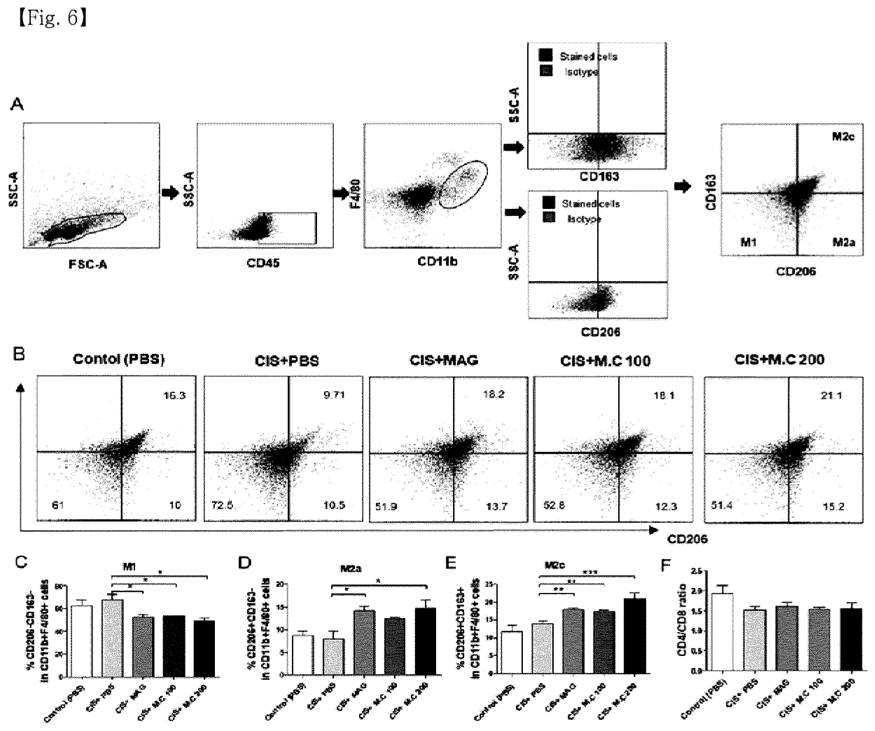

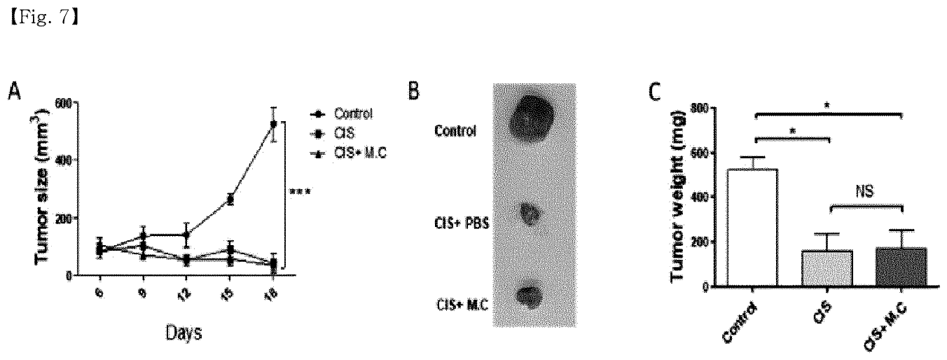

In an exemplary embodiment of the present disclosure, the Magnolia officinalis extract contains various ingredients in addition to the magnolol and honokiol (see FIG. 1), to have a significantly therapeutic effect on muscle disease compared to administration of magnolol alone at a concentration of more than 50 mg/kg, especially on muscle disease induced by administration of an anticancer drug.

In an exemplary embodiment, the composition may include a Magnolia officinalis extract at a concentration of 60 to 500 mg/kg.

In an exemplary embodiment, the composition may inhibit weight loss, muscle mass reduction, grip strength reduction, or muscle fiber damage.

As used herein, the term “extract” refers to an active ingredient isolated from a natural product, that is, a substance showing a desired activity. The extract may be obtained through an extraction process using water, an organic solvent, or a mixed solvent thereof, and includes dry powders of the extract or all forms formulated using the dry powders. In addition, the extract includes fractions obtained from the extract subjected to the extraction process. The extraction method of the extract is not particularly limited, and may be extracted by, for example, stirring extraction, shaking extraction, hot water extraction, cold immersion extraction, reflux cooling extraction, or ultrasonic extraction. The extraction solvent may be a polar solvent such as water, and lower alcohols having C₁-C₄, non-polar solvents such as hexane, chloroform, dichloromethane or ethyl acetate, or mixtures of two or more of these.

The composition of the present disclosure may be prepared as a pharmaceutical composition for prevention or treatment of muscle disease by further containing not only a Magnolia officinalis extract but also other effective ingredients having the same or similar function as or to the extract, or further containing other effective ingredients having different functions from the ingredients.

In an aspect, the present disclosure provides a pharmaceutical composition for prevention or treatment of anticancer drug side effect-induced disease, comprising a Magnolia officinalis extract as an active ingredient.

In an exemplary embodiment, the anticancer drug may be cyclophosphamide, methotrexate, 5-fluorouracil, doxorubicin, mustine, vincristine, procarbazine, prednisolone, bleomycin, vinblastine, dacarbazine, etoposide, cisplatin, epirubicin, cisplatin, capecitabine, or oxaliplatin, and more preferably cisplatin.

In an exemplary embodiment, the anticancer drug side effect-induced disease may be anticancer drug side effect-induced muscle disease, and more preferably at least one selected from the group consisting of muscular atrophy, muscle degeneration, muscle damage, muscular dystrophy, cachexia, and sarcopenia caused by the side effects of the anticancer drug, and more preferably cachexia or sarcopenia caused by the side effects of the anticancer drug.

In an exemplary embodiment, the composition may inhibit weight loss, muscle mass reduction, grip strength reduction, or muscle fiber damage caused by the anticancer drug.

In an exemplary embodiment, the composition may be administered separately, simultaneously, or sequentially from the anticancer drug.

In an exemplary embodiment, the pharmaceutical composition for prevention or treatment of the anticancer drug side effect-induced disease may be used as an anticancer adjuvant.

In an exemplary embodiment, the composition may promote the repair of muscle fibers damaged by the anticancer drug through macrophage polarization from M1 to M2.

As used herein, the term “prevention” refers to all actions that inhibit or delay the occurrence, spread, and recurrence of muscle disease or anticancer drug side effect-induced disease by administering the pharmaceutical composition according to the present disclosure. The “treatment” refers to all actions that improve or beneficially change the symptoms of muscle disease or anticancer drug side effect-induced disease by administering the composition of the present disclosure. Those skilled in the art to which the present disclosure pertains will be able to determine the degree of improvement, enhancement and treatment by knowing the exact criteria of disease for which the composition of the present disclosure is effective by referring to data presented by the Korean Academy of Medical Sciences, etc.

As used herein, the term “therapeutically effective amount” used in combination with the active ingredient means an amount effective to prevent or treat the muscle disease or anticancer drug side effect-induced disease, and the therapeutically effective amount of the composition of the present disclosure may vary depending on several factors, such as a method of administration, a target site, the condition of a patient, etc. Accordingly, when used in the human body, the dose should be determined as an appropriate amount in consideration of both safety and efficiency. It is also possible to estimate the amount used in humans from the effective amount determined through animal experiments. These matters to be considered when determining the effective amount are described in, for example, Hardman and Limbird, eds., Goodman and Gilman's The Pharmacological Basis of Therapeutics, 10th ed. (2001), Pergamon Press; and E.W. Martin ed., Remington's Pharmaceutical Sciences, 18th ed. (1990), Mack Publishing Co.

The pharmaceutical composition of the present disclosure is administered in a pharmaceutically effective amount. As used herein, the term “pharmaceutically effective amount” refers to an amount enough to treat disease at a reasonable benefit/risk ratio applicable to medical treatment and enough not to cause side effects. The effective dose level may be determined according to factors including a health condition of a patient, a type of muscle disease or anticancer drug side effect-induced disease, the occurrence cause and severity of muscle disease or anticancer drug side effect-induced disease, drug activity, sensitivity to drug, an administration method, an administration time, an administration route and excretion rate, a treatment period, and drugs used in combination or concurrently, and other factors well-known in medical fields. The composition of the present disclosure may be administered as an individual therapeutic agent or in combination with other therapeutic agents, and may be administered sequentially or simultaneously with conventional therapeutic agents, and may be administered singly or multiply. It is important to administer an amount capable of obtaining a maximum effect with a minimal amount without side effects by considering all the factors, which may be easily determined by those skilled in the art.

The pharmaceutical composition of the present disclosure may include carriers, diluents, excipients, or a combination of two or more thereof, which are commonly used in biological agents. As used herein, the term “pharmaceutically acceptable” means that the pharmaceutical composition exhibits non-toxic properties to cells or humans exposed to the composition. The carrier is not particularly limited as long as the carrier is suitable for in vivo delivery of the composition, and may be used by combining, for example, compounds described in Merck Index, 13th ed., Merck & Co. Inc., saline, sterile water, a Ringer's solution, buffered saline, a dextrose solution, a maltodextrin solution, glycerol, ethanol, and one or more of these ingredients, and if necessary, other conventional additives such as an antioxidant, a buffer, and a bacteriostat may be added. In addition, the pharmaceutical composition may be prepared in injectable formulations such as an aqueous solution, a suspension, and an emulsion, pills, capsules, granules, or tablets by further adding a diluent, a dispersant, a surfactant, a binder, and a lubricant. Furthermore, the pharmaceutical composition may be prepared preferably according to each disease or ingredient using a suitable method in the art or a method disclosed in Remington's Pharmaceutical Science (Mack Publishing Company, Easton PA, 18th, 1990).

In an exemplary embodiment, the pharmaceutical composition may be one or more formulations selected from the group including oral formulations, external formulations, suppositories, sterile injection solutions and sprays, and more preferably oral or injectable formulations.

As used herein, the term “administration” means providing a predetermined substance to a subject or patient by any suitable method, and the pharmaceutical composition may be administered parenterally (e.g., being applied as an injection formulation intravenously, subcutaneously, intraperitoneally or topically) or orally according to a desired method. The dose range may vary depending on the body weight, age, sex, and health condition of a patient, a diet, an administration time, an administration method, an excretion rate, the severity of a disease, etc. Liquid formulations for oral administration of the composition of the present disclosure correspond to suspensions, internal solutions, emulsions, syrups, etc., and may include various excipients, such as wetting agents, sweeteners, fragrances, preservatives, and the like, in addition to water and liquid paraffin, which are commonly used simple diluents. Formulations for parenteral administration include sterilized aqueous solutions, non-aqueous solvents, suspensions, emulsions, lyophilized agents, suppositories, and the like. The pharmaceutical composition of the present disclosure may also be administered by any device capable of transferring an active substance to a target cell. Preferred administration methods and formulations are intravenous injections, subcutaneous injections, intradermal injections, intramuscular injections, drop injections, etc. The injections may be prepared by using aqueous solvents such as a physiological saline solution and a ringer solution, and non-aqueous solvents such as vegetable oils, higher fatty acid esters (e.g., ethyl oleate), and alcohols (e.g., ethanol, benzyl alcohol, propylene glycol, glycerin, etc.). The injections may include pharmaceutical carriers, such as a stabilizer for the prevention of degeneration (e.g., ascorbic acid, sodium hydrogen sulfite, sodium pyrosulfite, BHA, tocopherol, EDTA, etc.), an emulsifier, a buffer for pH control, and a preservative to inhibit microbial growth (e.g., phenyl mercury nitrate, thimerosal, benzalkonium chloride, phenol, cresol, benzyl alcohol, etc.).

As used herein, the term “subject” refers to all animals including monkeys, cows, horses, sheep, pigs, chickens, turkeys, quails, cats, dogs, mice, rats, rabbits or guinea pigs including humans who have developed or may develop the muscle disease or anticancer drug side effect-induced disease, and the pharmaceutical composition of the present disclosure may be administered to a subject to effectively prevent or treat the diseases. The pharmaceutical composition of the present disclosure may be administered in combination with existing therapeutic agents.

The pharmaceutical composition of the present disclosure may further include a pharmaceutically acceptable additive. At this time, the pharmaceutically acceptable additive may be used with starch, gelatinized starch, microcrystalline cellulose, lactose, povidone, colloidal silicon dioxide, calcium hydrogen phosphate, lactose, mannitol, syrup, gum arabic, pregelatinized starch, corn starch, powdered cellulose, hydroxypropyl cellulose, Opadry, sodium starch glycolate, lead carnauba, synthetic aluminum silicate, stearic acid, magnesium stearate, aluminum stearate, calcium stearate, sucrose, dextrose, sorbitol, talc and the like. The pharmaceutically acceptable additive according to the present disclosure is preferably included in an amount of 0.1 part by weight to 90 parts by weight based on the composition, but is not limited thereto.

In an aspect, the present disclosure provides a food composition for prevention or alleviation of muscle disease comprising a Magnolia officinalis extract as an active ingredient.

In an exemplary embodiment, the muscle disease may be muscle disease caused by cancer or muscle disease caused by anticancer drug treatment.

In an exemplary embodiment, the muscle disease may be at least one selected from the group consisting of muscular atrophy, myopathy, muscular degeneration, myasthenia, muscular injury, dystrophinopathy, myopathy, muscular dystrophy, cachexia, and sarcopenia.

When the composition of the present disclosure is used as the food composition, the composition may be added as it is or used with other foods or food ingredients, and may be appropriately used according to a general method. The composition may include food acceptable supplement additives in addition to the active ingredients, and the mixing amount of the active ingredients may be appropriately determined depending on the purpose of use (prevention, health or therapeutic treatment).

As used herein, the term “food supplement additive” means a component that may be supplementally added to food, and may be appropriately selected and used by those skilled in the art as being added to prepare a health functional food of each formulation. Examples of the food supplement additives include various nutrients, vitamins, minerals (electrolytes), flavors such as synthetic and natural flavors, colorants and fillers, pectic acid and salts thereof, alginic acid and salts thereof, organic acids, protective colloidal thickeners, pH adjusters, stabilizers, preservatives, glycerin, alcohols, carbonation agents used in carbonated drinks, and the like, but the types of food supplement additives of the present disclosure are not limited by the examples.

The food composition of the present disclosure may include a health functional food. As used herein, the term “health functional food” refers to food prepared and processed in the form of tablets, capsules, powders, granules, liquids and pills by using raw materials or ingredients having functionalities useful to the human body. Here, the ‘functionality’ means regulating nutrients to the structure and function of the human body or obtaining effects useful for health applications such as physiological action. The health functional food of the present disclosure is able to be prepared by methods to be commonly used in the art and may be prepared by adding raw materials and ingredients which are commonly added in the art in preparation. In addition, the formulations of the health functional food may also be prepared with any formulation recognized as a health functional food without limitation. The food composition of the present disclosure may be prepared in various types of formulations, and unlike general drugs, the food composition has an advantage that there is no side effect that may occur when taking a long-term use of the drug by using the food as a raw material, and has excellent portability, so that the health functional food of the present disclosure may be taken as supplements to enhance the effects of anticancer drugs.

In addition, there is no limitation in types of health food in which the composition of the present disclosure may be used. In addition, the composition including the Magnolia officinalis extract of the present disclosure as the active ingredient may be prepared by mixing known additives with other suitable auxiliary ingredients that may be contained in the health functional food according to the selection of those skilled in the art. Examples of foods to be added include meat, sausage, bread, chocolate, candy, snacks, confectionery, pizza, ramen, other noodles, gum, dairy products including ice cream, various soups, beverages, tea, drinks, alcoholic beverages, vitamin complexes and the like, and may be prepared to be added to extract, tea, jelly, juice, and the like prepared by using the extract according to the present disclosure as a main ingredient.

In an aspect, the present disclosure provides a composition for promoting differentiation of anti-inflammatory macrophages including a Magnolia officinalis extract as an active ingredient.

In an exemplary embodiment, the composition may induce M2 macrophage polarization and increase the expression of like growth factor-1 (IGF-1).

In an exemplary embodiment, the composition may decrease a M1 macrophage (CD206⁺CD163⁻) group and increase an M2a macrophage (CD206⁺CD163⁻) group and an M2c macrophage (CD206⁺CD163⁺) group.

In an aspect, the present disclosure provides a method for treating muscle disease including administering a Magnolia officinalis extract in a pharmaceutically effective amount to a subject suffering from the muscle disease.

In an aspect, the present disclosure provides a use of a Magnolia officinalis extract for use in the preparation of a pharmaceutical composition for prevention and treatment of muscle disease.

In an aspect, the present disclosure provides a method for treating anticancer drug side effect-induced disease including administering a Magnolia officinalis extract in a pharmaceutically effective amount to a subject suffering from the anticancer drug side effect-induced disease.

In an exemplary embodiment, the anticancer drug side effect-induced disease may be muscle disease, and more preferably at least one selected from the group consisting of muscular atrophy, muscular degeneration, muscle damage, muscular dystrophy, cachexia and sarcopenia.

In an aspect, the present disclosure provides a use of a Magnolia officinalis extract for use in the preparation of a pharmaceutical composition for prevention and treatment of anticancer drug side effect-induced disease.

### MODES OF THE INVENTION

Hereinafter, the present disclosure will be described in more detail with reference to the following Examples. However, the following Examples are only intended to embody the contents of the present disclosure, and the present disclosure is not limited thereto.

### Example 1. Preparation of Magnolia Officinalis Extract

1000 mL of an ethanol:water (7:3, v/v) mixed solvent (70% ethanol) was mixed with 200 g of Magnolia officinalis (Magnolia officinalis Rehder et Wilson) (Sunchen, Korea), extracted twice in a reflux system at 70° C., and then filtered. Thereafter, the extract was concentrated under reduced pressure at 40 to 45° C. and freeze-dried for 3 days to produce a Magnolia officinalis extract (M.C) with a yield of 10.91% (21.82 g).

### Example 2. Ingredient Analysis of Magnolia Officinalis Extract

The ingredients of the Magnolia officinalis ethanol extract prepared in Example 1 were analyzed using HPLC, and magnolol and honokiol present in the Magnolia officinalis ethanol extract were analyzed at the Dong-eul Herbal Medicine Analysis Center (Busan, Korea). Specifically, octadecyl silica (ODS)-chromatographic analysis of magnolol and honokiol was performed using a DHAC-12-01 (Agilent, Santa Clara, CA, USA) equipped with a UV-vis spectrophotometer (289 nm). The magnolol and honokiol were analyzed by an isocratic elution with acetonitrile:distilled water:acetic acid (70:30:1, v/v/v) as a mobile phase component using an OSAKASODA C18 column (5 μm particle size, 4.6×250 mm; Osaka, Japan) at 20° C., and an injection volume and flow rate were 10 μL and 0.3 mL/min, respectively. The magnolol and honokiol standards were detected at 289 nm for quantitative HPLC analysis. In addition, the freeze-dried powder (0.1 g) of the Magnolia officinalis ethanol extract prepared in Example 1 was dissolved in methanol (100 mL), sonicated for 20 minutes, filtered, and then used for ODS-HPLC analysis. In addition, the magnolol and honokiol standards were each dissolved in 100% methanol at a concentration of 100 mg/L and HPLC analysis was performed.

As a result, it was confirmed that the contents of magnolol and honokiol in the Magnolia officinalis ethanol extract (M.C) were 2.1% and 0.8%, respectively (FIG. 1).

### Example 3. Effect of Magnolia Officinalis Extract on Inhibiting Cisplatin-Mediated Weight Loss

To determine what a Magnolia officinalis extract had any effect on muscle atrophy and rapid weight loss induced by cisplatin, which was a common chemotherapy anticancer drug, mice were administered with cisplatin to induce weight loss, and administered with the Magnolia officinalis ethanol extract (M.C) of Example 1, and then the effect thereof was confirmed. Specifically, mice (C57BL/6, 7 to 8 weeks old) (Jackson Laboratories, Raon Bio, Gyeonggi) were randomly caged and raised in a regulated environment with free access to food and water with a 12-hour light/dark cycle, and then on days 1 to 5 and days 26 to 30, cisplatin (Sigma-Aldrich, P4394; St. Louis, MO, USA) (2.5 mg/kg) dissolved at 1 mg/mL in physiological saline was injected intraperitoneally once daily (sarcopenia mouse model), M.C (50, 100 and 200 mg/kg) were administered orally every 3 days (total of 12 times). In addition, a control group (control) and a negative control group (CIS+PBS) were orally administered with PBS every 3 days, and a positive control group was injected intraperitoneally every 3 days (total of 12 times) with magnolol (purity≥95%) (Sigma (m3445; Sigma-Aldrich, St. Louis, MO, USA) (10 mg/kg) dissolved in DMSO at 10 mM. At this time, all experimental processes were conducted with approval from Kyung Hee Institutional Animal Care (KHUASP (SE)-20-529). As a result, administration of cisplatin induced rapid weight loss in mice from day 15, which was significantly alleviated by administration of M.C or magnolol, resulting in rapid weight recovery (FIG. 2). Such a significant difference between the CIS (cisplatin)+PBS administered group and the CIS+M.C administered group persisted for 6 days or more, and the weight of the mice decreased by 30% or more, and then the experiment was terminated on day 42 (A of FIG. 2). In particular, it was shown that a group administered with M.C at 100 or 200 mg/kg significantly inhibited weight loss caused by cisplatin compared to a magnolol administered group. In addition, as a result of confirming changes in weight on the last and first days, mice administered with cisplatin showed significant weight gain, whereas the group administered with M.C or magnolol showed a significant reduction in weight loss induced by cisplatin (B of FIG. 2). To determine whether such weight loss was associated with digestion and loss of appetite, colon length and average daily food uptake were measured, and as a result, it was shown that there was no difference in colon length and food uptake between the groups (C and D of FIG. 2), and thus cisplatin-induced weight loss was not related to abnormal digestion or appetite changes.

### Example 4. Example 4. Effect of Magnolia Officinalis Extract on Alleviating Cisplatin-Skeletal Muscle Wasting

### 4-1. Confirmed Effect of Inhibiting Muscle Mass Reduction

In order to confirm an effect of a Magnolia officinalis extract on cisplatin-induced skeletal muscle wasting, the leg weight and tibialis anterior (TA) muscle mass of each group were measured on day 42 of the experiment. As a result, both the M.C administered group and the magnolol administered group significantly alleviated the leg weight and TA muscle mass reduction induced by cisplatin, and it was shown that such an effect was significantly higher in the M.C administered group than the magnolol administered group (A to C of FIG. 3).

### 4-2. Confirmed Effect of Inhibiting Grip Strength Reduction

To determine the effect of the Magnolia officinalis extract on cisplatin-induced grip strength reduction, muscle strength of all limbs and muscle strength of forelimb were measured using a digital force gauge (DS2-5N; IMADA Inc., Northbrook, IL, USA). The muscle strength was measured by pulling the mouse's tail downward 5times and recording the peak tension when the mouse released its foot, and repeated 3 times at 10-minute intervals, and the average of 5 values was used for calculation. As a result, it was shown that the pulling force power was significantly increased in the M.C administered group (100 and 200 mg/kg) and the magnolol administered group compared to the cisplatin administered group, and it was shown that this effect was significantly higher in the group administered with M.C at 100 mg/kg or 200 mg/kg than in the magnolol administered group (D of FIG. 3).

### 4-3. Confirmed Effect of Inhibiting Muscle Fiber Damage

In order to confirm an effect of a Magnolia officinalis extract on cisplatin-induced muscle fiber damage, the muscle fiber morphology of skeletal muscle was confirmed through hematoxylin and eosin (H&E) staining. Specifically, TA muscle tissue sections were fixed in 4% formalin for 24 hours, embedded in paraffin, and sectioned at a thickness of 4 μm. The sectioned sections were processed as described in Lee et al., H&E staining was performed to evaluate tissue pathological changes, and imaged with an Eclipse Ci-L microscope (Nikon, Tokyo, Japan). As a result, unlike the group administered with 50 mg/kg of M.C, muscle fiber deformation (muscle fiber damage) was inhibited in both groups administered with 100 or 200 mg/kg (E of FIG. 3), and thus it was confirmed that the Magnolia officinalis extract prevented or inhibited muscle loss or damage in mice developed with cisplatin-induced sarcopenia.

### Example 5. Effect of Activating M1 and M2 Macrophage Polarization in Skeletal Muscle

Since the repair of damaged muscle fibers requires activation of M2 macrophages, it was confirmed whether the expression of macrophage-specific marker genes, including Arg-1, NOS2, TNF-α, MRC1, TGF-β, and CD163 was changed in skeletal muscle by administration of the Magnolia officinalis extract using real-time quantitative PCR (qPCR). Specifically, total RNA was extracted from TA muscle using an easy-BLUE TM Total RNA Extraction Kit (17061; iNtRON, Biotechnology, Jungwon, Korea), and then cDNA was synthesized using a Maxime RT-PCR PreMix Kit (25131; iNtRON Biotechnology, Jungwon, Korea). Thereafter, real-time qPCR was performed using a primer set (5′->3′) for each gene below and a SensiFAST™SYBR No-ROX Kit (BIO-98020; Medison bioline, Roma, Italia), and each gene was analyzed using a standard 2^{δδCt} method and normalized with glyceraldehyde-3-phosphate dehydrogenase (GAPDH): Nos2 (Forward: CAC CTT GGA GTT CAC CCA GT (SEQ ID NO: 1), Reverse: ACC ACT CGT ACT TGG GAT GC (SEQ ID NO: 2)), Igf-1 (Forward: CTA CCA AAA TGA CCG CAC CT (SEQ ID NO: 3), Reverse: CAC GAA CTG AAG AGC ATC CA (SEQ ID NO: 4)), Tgfb1 (Forward: CAA GGA AGG TTG GCA TTT GT (SEQ ID NO: 5), Reverse: AGG TAA CGC CAG GAA TTG CA (SEQ ID NO: 6)), Tnfa (Forward: GCT GAG CTC AAA CCC TGG TA (SEQ ID NO: 7), Reverse: CCG GAC TCC GCA AAGTCT AA (SEQ ID NO: 8)), Mrc1 (Forward: CAA GGA AGG TTG GCA TTT GT (SEQ ID NO: 9), Reverse: CCT TTC AGT CCT TTG CAA GC (SEQ ID NO: 10)), Arg-1 (Forward: GGC TGG TCT GCT TGA GAA AC (SEQ ID NO: 11), Reverse: CTT TTC CCA CAG ACC TTG GA (SEQ ID NO: 12)), Cd163 (Forward: TGG TGT GCA GGG AAT TAC AA (SEQ ID NO: 13), Reverse: ATC CCT GCT GTG GGT ACA AG (SEQ ID NO: 14)) and Gapdh (Forward: ACC CAG AAG ACT GTG GAT GG (SEQ ID NO: 15), Reverse: CAC ATT GGG GGT AGG AAC AC (SEQ ID NO: 16)).

As a result, the expression of M1 macrophage-specific marker genes TNF-α and iNOS increased in the cisplatin administered group, but was significantly decreased by M.C treatment (A and B of FIG. 4). At the same time, it wash shown that the expression levels of M2 macrophage-specific marker genes CD206, Arg-1, TGF-β, and CD163 were significantly increased in the M.C administered group compared to the cisplatin administered group (C to F of FIG. 4). Through this, it was confirmed that the Magnolia officinalis extract regulated M1 and M2 macrophage polarization in a skeletal muscle microenvironment.

### Example 6. Effect of Increasing Number of Macrophages and IGF-1 Expression in Skeletal Muscle

### 6-1. Confirmation of Number of Macrophages in Skeletal Muscle

It was confirmed whether like growth factor-1 (IGF-1), which shifted the polarization of a macrophage phenotype from M2a to M2c (macrophages involved in extracellular matrix remodeling and muscle healing), and the number of macrophages were affected in skeletal muscle by administration of a Magnolia officinalis extract. Specifically, the TA muscle tissue sections of Example 4-3 were incubated overnight with a primary antibody, rat anti-mouse CD68 antibody (1:250; MCA1957GA; Bio-Rad, Contra Costa County, CA, USA) and rabbit anti-mouse IGF-1 antibody (1:500; 40657; Abcam, Cambridge, UK), and then incubated for 30 minutes with a secondary antibody, Alexa Fluor 488-conjugated goat anti-rat immunoglobulin IgG (A11006; Invitrogen, Carls-bad, CA, USA) and Alexa Fluor 594-conjugated goat anti-rabbit IgG (A32740; Invitrogen, Carlsbad, CA, USA). Thereafter, the sections were mounted using mounting media containing 4′,6-diamidino-2-phenylindole (DAPI) (H-1200, Vector, Torrance, CA, USA), and then imaged under a confocalator microscope (FV10C-PSU; Olympus Corporation, Tokyo, Japan). In the captured images, the numbers of IGF-I (red), CD68 (macrophage) (green), and DAPI (blue) positive cells per image area were measured in at least five random fields.

As a result, it was shown that the number of cells expressing both CD68 and IGF-1 increased in both the M.C and magnolol-administered groups (A and B of FIG. 5).

### 6-2. Confirmation of IGF-1 Expression in Skeletal Muscle

The expression levels of IGF-1 mRNA in the TA muscle tissue sections of Example 4-3 were confirmed by qPCR, and the expression levels of IGF-1 protein were confirmed by ELISA. Specifically, qPCR was performed as in Example 5, and ELISA was performed by dissolving TA muscle tissue sections using a PRO-PREP™ Protein Extraction Solution (17081; iNtRON Biotechnology, Jungwon, Korea) containing a protease inhibitor cocktail and then homogenizing the sections using a mechanical homogenizer (Precellys R24; Bertin, Montigny-le-bretonneux, France). Thereafter, ELISA analysis was performed using an ELISA kit for IGF-1 (DY791; R&D Systems, Minneapolis, MN, USA).

As a result, it was shown that the expression of both mRNA and protein of IGF-1 was significantly increased in the M.C administered group compared to the cisplatin administered group (C and D of FIG. 5).

Through this, it was confirmed that the Magnolia officinalis extract increased the expression of macrophage-derived IGF-1 in skeletal muscle to shift polarization to M2a and M2c phenotypes, thereby promoting muscle recovery from cisplatin-induced skeletal muscle damage and protecting against muscular dystrophy.

### Example 7. Effect of Macrophage Subtype Change

In order to confirm an effect of a Magnolia officinalis extract on the changes in M1 macrophage phenotype, spleen-derived macrophages were extracted from each group in the sarcopenia mouse model of Example 3, and then the subtypes of the macrophages were confirmed by flow cytometry. Specifically, the spleens of mice in each group were extracted and then separated into single cells using a 40 μm nylon mesh strainer. Thereafter, red blood cells were lysed with a Pharmlyse buffer (555899; BD bioscience, San Jose, CA, USA), and single cells were incubated and stained with anti-mCD8 APC-CY7 antibody (100713; BioLegend, San Diego, CA, USA), anti-mCD4 APC antibody (100412; BioLegend, San Diego, CA, USA), anti-mCD45 Pacific Blue (MCD4528; Invitrogen, Carlsbad, CA, USA), anti-mF4/80 BV421 antibody (123131; BioLegend, San Diego, CA, USA), anti-mCD11b PerCP-Cy 5.5 antibody (101227; BioLegend, San Diego, CA, USA), anti-mCD163 PE antibody (12-1631-82; e-bioscience; Thermo Fisher Scientific, Middlesex County, MA, USA) and anti-mCD206 APC antibody (141707; BioLegend, San Diego, CA, USA) at 4° C. for 1 hour. The stained cells were washed twice with a FACS buffer and then analyzed using FACS Lyric instruments (BD bioscience, San Jose, CA, USA). Data were analyzed by Flow Jo software (Treestar, Inc., San Carlos, CA, USA). Unstained cells (negative control group) were used as a gating control group. The cell phenotypes were as follows (A of FIG. 6): Among cells gated on CD45⁺ and CD11b⁺F4/80⁺, M1 macrophages were CD206⁻CD163⁻ cells; M2a macrophages were CD206⁺CD163⁻ cells; and M2c macrophages were CD206⁺CD163⁺ cells.

As a result, an M1 macrophage (CD206⁻CD163⁻) group within CD11b⁺F4/80⁺ macrophages was significantly reduced by treatment of the Magnolia officinalis extract compared to the cisplatin administered group (B and C of FIG. 6). In addition, the M2a macrophage (CD206⁺CD163⁻) and M2c macrophage (CD206⁺CD163⁺) groups were significantly increased in the Magnolia officinalis extract administered group (D and E of FIG. 6). In addition, it was shown that as a result of measuring the CD4⁺/CD8⁺ T cell ratio, which indicated changes in T cell subgroups, there was no difference between the groups (F of FIG. 6).

### Example 8. Confirmation of Effect of Magnolia Officinalis Extract on Anti-Tumor Activity of Cisplatin

To confirm whether administration of the Magnolia officinalis extract interfered with the anti-tumor activity of cisplatin, a mouse colon cancer cell line CT26 (Korean Cell Line Bank, 80009; Seoul, Korea) was injected subcutaneously at 3×10⁵ per mouse to fabricate a colon cancer-bearing mouse model, and then treated with cisplatin or cisplatin+Magnolia officinalis extract (200 mg/kg), and the size (volume) and weight of tumor were measured every 3 days. As a result, cisplatin significantly reduced tumor growth, and even in the group administered with the Magnolia officinalis extract and cisplatin, the tumor growth inhibition effect was similar to the tumor growth inhibition effect caused by administration cisplatin alone (FIG. 7), and thus it was confirmed that the Magnolia officinalis extract did not interfere with the anti-tumor function of cisplatin.

As a result, it was confirmed that the Magnolia officinalis extract had a preventive and therapeutic effect on cancer cachexia, especially cisplatin-induced sarcopenia, and did not interfere with a cisplatin-induced anti-tumor effect.

1. A method for preventing or treating a muscle disease, the method comprising administering to a subject in need thereof a pharmaceutical composition comprising a Magnolia officinalis extract as an active ingredient.

2. The method of claim 1, wherein the muscle disease is caused by decreased muscle function, muscle tissue damage, muscle wasting, or muscle degeneration.

3. The method of claim 1, wherein the muscle disease is caused by cancer.

4. The method of claim 1, wherein the muscle disease is at least one selected from the group consisting of muscular atrophy, myopathy, muscular degeneration, myasthenia, muscular injury, dystrophinopathy, myopathy, muscular dystrophy, cachexia, and sarcopenia.

5. The method of claim 1, wherein the Magnolia officinalis extract is extracted with at least one solvent selected from the group consisting of water, organic solvents, subcritical fluids and supercritical fluids.

6. The method of claim 1, wherein the Magnolia officinalis extract comprises magnolol and honokiol in a ratio of 2:1 to 4:1 (w/w).

7. The method of claim 1, wherein the Magnolia officinalis extract is comprised at a concentration of 60 to 500 mg/kg.

8. The method of claim 1, wherein the subject require inhibiting weight loss, muscle mass reduction, grip strength reduction, or muscle fiber damage.

9. A method for preventing or treating an anticancer drug side effect-induced disease, the method comprising administering to a subject in need thereof a pharmaceutical composition comprising a Magnolia officinalis extract as an active ingredient.

10. The method of claim 9, wherein the anticancer drug is cyclophosphamide, methotrexate, 5-fluorouracil, doxorubicin, mustine, vincristine, procarbazine, prednisolone, bleomycin, vinblastine, dacarbazine, etoposide, cisplatin, epirubicin, cisplatin, capecitabine, or oxaliplatin.

11. The method of claim 9, wherein the anticancer drug side effect-induced disease is at least one selected from the group consisting of muscular atrophy, muscle degeneration, muscle damage, muscular dystrophy, cachexia, and sarcopenia.

12. The method of claim 9, wherein the subject requires inhibiting anticancer drug-induced weight loss, muscle mass reduction, grip strength reduction, or muscle fiber damage.

13. The method of claim 9, wherein the pharmaceutical composition is administered separately, simultaneously, or sequentially from the anticancer drug.

14-21. (canceled)
